(19) United States
(12) Patent Application Publication
(10) Pub. No.: US 2024/0268903 A1
(43) Pub. Date: Aug. 15, 2024

(54) SYSTEM AND METHOD FOR CONFIGURING AT LEAST ONE MEDICAL DEVICE

(71) Applicant: CARL ZEISS MEDITEC AG, Jena (DE)

(72) Inventors: Alexander SCHENK, Frankehardt (DE); Akhil RAMESH KUMAR, Oberkochen (DE); Johanna SCHMIDT, Oberhaching (DE); Sebastian KASSNER, München (DE)

(21) Appl. No.: 18/693,512

(22) PCT Filed: Sep. 15, 2022
(86) PCT No.: PCT/DE2022/100687

§ 371 Date: Mar. 19, 2024

(30) Foreign Application Priority Data
Sep. 20, 2021 (DE) 10 2021 210 397.9

(51) Int. Cl. A61B 34/00; A61B 17/00; G16H 40/63

(52) U.S. Cl. CPC A61B 34/25; A61B 17/00; G16H 40/63; A61B 2017/00367; A61B 2034/252; A61B 2034/254; A61B 2034/258

(57) ABSTRACT

A system for configuring at least one medical device is disclosed, wherein the system comprises: a display unit designed to display predefined steps for at least one medical process, the predefined steps including instructions for a surgeon and settings of the at least one medical device, a transceiver unit designed to receive an input by the surgeon prior to the medical process, and a processing unit designed to adapt the predefined steps on the basis of the input by the surgeon, the adaptation including a selection of the predefined steps, a sequence of the predefined steps and/or parameters for the settings of the at least one medical device.

### CROSS-REFERENCE TO RELATED APPLICATIONS

The present application is the U.S. National Stage entry of International Application No. PCT/DE2022/100687, filed Sep. 15, 2022, which claims priority under 35 U.S.C. § 119 to German Patent Application No. 10 2021 210 397.9, filed Sep. 20, 2021, the contents of which are incorporated by reference herein in their entirety.

The present invention relates to a system for configuring at least one medical device. The present invention also relates to a method for configuring at least one medical device.

Medical processes, for example operations, usually consist of various steps, which describe the medical process or procedure and are carried out in succession. Especially in the case of operations, for example eye operations, e.g. cataract operations, which are performed with the aid of various medical devices such as microscopes, phacoemulsification systems, etc., the various steps may require different settings for the required medical devices in each case. Current practice has a surgeon adapt the settings of the medical devices for the respective current operation step during the operation. However, this lengthens the operation duration on account of the time-consuming readjustments.

It is therefore the object of the present invention to provide a system and a method enabling an efficient execution of such operations.

This object is achieved by a system for configuring at least one medical device, as disclosed herein, and by a method for configuring at least one medical device, as disclosed herein.

The system for configuring at least one medical device comprises a display unit designed to display predefined steps for at least one medical process, the predefined steps including instructions for a surgeon and settings of the at least one medical device.

As already explained above, a medical process, for example a cataract operation, usually consists of multiple steps describing the procedure. In this context, a medical process has predefined steps which are defined in general terms and for example contain instructions for the surgeon. Such instructions may be acoustic or visual directions or visualizations which relate to, in particular, the medical device used and the current step of the medical process. These generally predefined steps also contain settings of the at least one medical device. If a step of the medical process requires multiple medical devices, then the corresponding predefined steps contain settings for multiple medical devices. For example, medical devices may include microscopes, phacoemulsification systems (including handpieces), endoscopes, diagnostic devices, etc. Moreover assistance apparatuses, including cloud-based functionalities, may also be used as medical devices in the broadest sense, with the aid of which, for example, the steps required for the medical process are facilitated. According to the invention, these devices can also be set in advance.

These steps predefined in general terms contain many instructions and settings possible in theory, especially as default values for the at least one medical device. However, a specific process related to a certain patient may require only some of the predefined steps and specific settings of the at least one medical device, which go beyond the general default settings of the predefined steps.

So as to now avoid time-consuming adjustments during the medical procedure and design the medical procedure or process efficiently, the system described here comprises a transceiver unit designed to receive an input by the surgeon prior to the medical process. The input by the surgeon serves to personalize the medical process, and in particular adapt the latter to a patient. Since this occurs in advance, prior to the actual operation, it is possible to avoid the situation where adjustments to the at least one medical device become necessary during the operation and where predefined steps not required during the actually performed operation are displayed.

The system also comprises a processing unit designed to adapt the predefined steps on the basis of the input by the surgeon. In particular, the adaptation of the predefined steps contains a selection of the predefined steps. This means that the surgeon is able to select those steps from the totality of predefined steps which are required for the specific medical process. Moreover, the adaptation may contain the adaptation of a sequence of the predefined steps. Thus, in addition to selecting predefined steps, the surgeon is also able to adapt the sequence of the predefined steps if a different sequence of the steps is required or preferable during the specific operation. Moreover, the adaptation may include parameters for the settings of the at least one medical device. In the individual predefined steps, the necessary parameters are chosen automatically on one or more medical devices. The adaptation allows these parameters to be adapted such that they are adapted to the medical process to be performed currently and to the corresponding patient. For example, such parameters may include an irrigation pressure, an aspiration pressure, a diathermy power, an ultrasound power, a brightness of the endo-light source(s), an air output pressure and/or an oil pressure, to name but a few.

In other words, the predefined steps contain default values and are based on a default sequence for the at least one medical device, and these default values and/or the default sequence can be adapted on an individual basis by the surgeon for the specific medical process. Moreover, the predefined steps for a specific medical process can be renamed by the input by the surgeon.

Thus, the system described here renders it possible to already adapt predefined steps for a medical process, in particular adapt these patient specifically, in advance, i.e. prior to the operation itself. This can improve the efficiency during the operation since the surgeon need implement fewer or no adjustments on the medical devices during the operation.

According to an embodiment, the processing unit is also designed to work through the adapted steps in sequence during the medical process and, on the basis of the adapted steps, output instructions for the surgeon and configure the settings of the at least one medical device for each adapted step. In this case, the instructions may include directions, especially audio outputs, or visualizations for the surgeon, which may be output on different devices. This output can be implemented on the display unit or on one or more medical devices. The display unit or output unit may also be implemented on one or more medical devices. Moreover, the instruction itself may also contain information about the currently used step.

In addition to the instructions for the surgeon, the settings of each adapted step are transferred to the at least one medical device or, in the case of multiple medical devices, to each medical device, in order to implement the appropriate settings there. This point can be dispensed with if no changes in the settings are required in one of the adapted steps.

The processing of the predefined steps by way of the processing unit can be implemented on a mobile device, a local device, for example the medical device or a computer, and/or in the form of a cloud service or the like. The adapted steps can be transferred to the medical device before the start of the operation and can be displayed and/or executed there during the operation (in particular the configuration of the medical device for each step). Rather than transferring all adapted steps to the medical device before the operation, it is also possible that the processing unit only transfers the required settings/instructions to the surgeon and/or the medical device when working through the adapted steps during the medical process.

In particular, the transceiver unit can be designed to receive an input by the surgeon, retrieve the predefined steps from a storage unit, transfer the settings to the at least one medical device, and/or can be designed for other communications processes required for the configuration of the at least one medical device. The transceiver unit can communicate wirelessly or in wired fashion with the various units or devices. Likewise, the transceiver unit can communicate with the various units or devices directly or indirectly, for example via a server and/or a cloud. Moreover, the system may be designed as an overall system implemented as a single apparatus, or it may be implemented as a distributed system, with the individual units being implemented on various apparatuses, for example local or remotely arranged computing units/devices and/or servers.

According to a further embodiment, the transceiver unit is designed to receive an input by the surgeon during the medical process. In that case, the processing unit may be moreover designed to further adapt, on the basis of the input by the surgeon, the steps adapted prior to the medical process, the adaptation including a selection of the adapted steps, a sequence of the adapted steps and/or parameters for the settings of the at least one medical device. Moreover, the input by the surgeon during the medical process may bring about a changeover between the adapted steps. In addition to the adaptation of the predefined steps in advance, i.e. prior to the operation, the surgeon, according to this embodiment, may also undertake a further adaptation of the steps during the medical process. Thus, should it transpire during the operation that the steps adapted in advance require a further adaptation, for example on account of unexpected complications, the surgeon may undertake this adaptation during the operation as well. Moreover, the surgeon can manually introduce a changeover between the adapted steps, for example if they establish during the operation that a step is not required or has already been carried out. For example, a changeover may also be initiated if a step is intended to be shortened. In this case, a step can be terminated/shortened by the input by the surgeon and there can be a direct changeover to the next step.

As already explained above, the adaptation of the predefined steps can be patient specific in particular. Moreover, the predefined steps may be defined in general for all operations and may not be adapted to a specific type of operation (e.g. cataract operation). In this case, the surgeon can adapt the predefined steps operation-specifically, preferably in combination with a patient-specific adaptation.

According to a further embodiment, the display unit comprises a graphical user interface designed to display the predefined steps, the adapted steps, the settings of the at least one medical device and/or the instructions for the surgeon. The graphical user interface offers a simple and intuitive option for adapting the predefined steps and displaying the further information, for instance the settings or the instructions. Consequently, the surgeon does not require a more in-depth understanding of the system; instead, they can simply process or work through the steps of the medical process, known to them, via the graphical user interface.

According to a further embodiment, the display unit is designed to output the instructions for the surgeon visually and/or acoustically. Depending on the medical device used, the display unit can visually output the instructions, for example on the medical device, e.g. in the form of an overlay in an image. In an alternative or in addition, the instructions may also be output acoustically, for example as a speech output. The display unit can display the adapted steps and/or the instructions for the surgeon in succession during the medical process. In an alternative, the adapted steps and/or the instructions for the surgeon can be displayed simultaneously, and the respective active step is highlighted.

According to a further embodiment, the transceiver unit comprises one or more input devices, in particular a microphone, a mouse, a keyboard, a touchpad and/or a foot switch. The transceiver unit may also be connected to one or more such input devices, wherein these may for example be part of the medical device or of a further apparatus or device. During the medical process, the surgeon may consider a foot switch, in particular, to be advantageous, especially for performing a changeover between the adapted steps. Such a foot switch is easy to operate, especially during an operation. Instead of a foot switch, it is for example also possible for the individual steps to be selected or changed at the medical device, for example a microscope, by way of an operating button or the like.

According to a further embodiment, the system comprises at least one storage unit designed to store the predefined steps and/or the adapted steps with the associated instructions and settings. For example, the at least one storage unit may be arranged on the at least one medical device and/or on a server. As already mentioned above, the transceiver unit may communicate wirelessly or in wired fashion with the storage unit in order to transfer the data to be stored or retrieved. Prior to being processed by the surgeon, the predefined steps can be retrieved from the storage unit by the transceiver unit. In particular, the adapted steps can be stored as a result of a surgeon input.

According to a further aspect, a method for configuring at least one medical device is proposed. The method comprises the following steps: displaying predefined steps for at least one medical process, the predefined steps including instructions for a surgeon and settings of the at least one medical device, receiving an input by the surgeon prior to the medical process, and adapting the predefined steps on the basis of the input by the surgeon, the adaptation including a selection of the predefined steps, a sequence of the predefined steps and/or parameters for the settings of the at least one medical device.

The embodiments and features described for the proposed system are correspondingly applicable to the proposed method.

Further, a computer program product is proposed, the latter having a program code designed to prompt the execution of the method as explained above on a computer.

A computer program product, for example a computer program medium, can be provided or supplied, for example, as a storage medium, for example a memory card, a USB stick, a CD-ROM, a DVD, or else in the form of a downloadable file from a server in a network. By way of example, in a wireless communications network, this can be effected by transferring an appropriate file with the computer program product or the computer program means.

Further possible implementations of the invention also comprise not explicitly mentioned combinations of features or embodiments described above or below with respect to the exemplary embodiments. In this case, a person skilled in the art will also add individual aspects as improvements or supplementations to the respective basic form of the invention.

Further advantages and advantageous embodiments are specified in the description, the drawings and the claims. In this case, the combinations of the features specified in the description and in the drawings in particular are purely exemplary, and so the features may also be present individually or in other combinations.

The intention is to describe the invention in detail below on the basis of exemplary embodiments depicted in the drawings. In this case, the exemplary embodiments and the combinations shown in the exemplary embodiments are merely exemplary and are not intended to define the scope of protection of the invention. The latter is defined purely by the appended claims.

### IN THE FIGURES

FIG. 1 shows a schematic block diagram of a system for configuring a medical device; and

FIG. 2 shows a schematic flowchart of a method for configuring a medical device.

Hereinbelow, the same or functionally equivalent elements are labeled using the same reference signs.

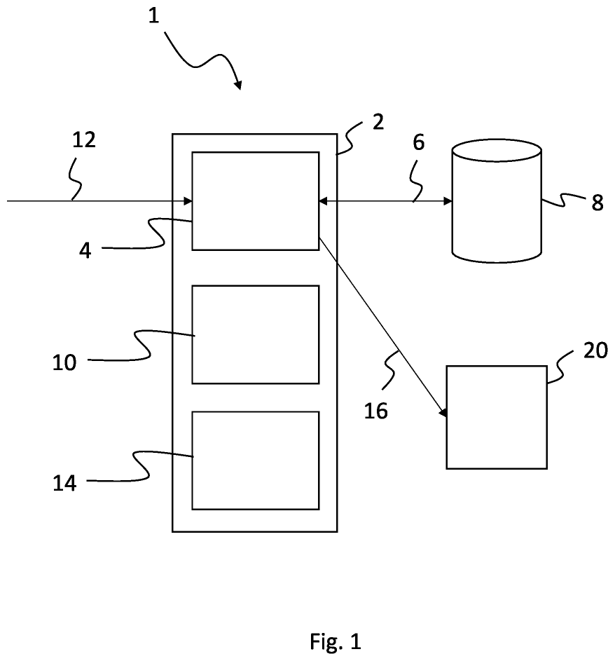

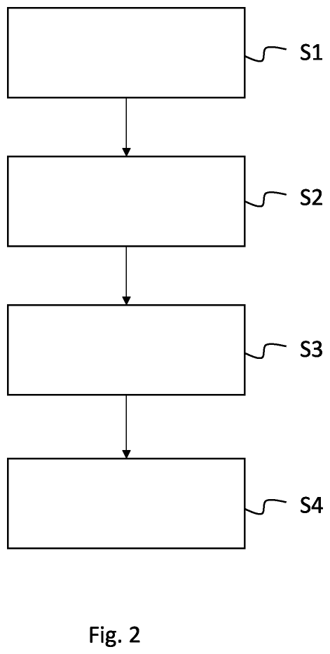

FIG. 1 shows a system 1 for configuring at least one medical device 20. Even though only one medical device 20 is shown, two or more medical devices 20 can also be configured. For example, the medical device 20 can be part of a phacoemulsification system, a microscope, an endoscope or the like. Various adjustments of the medical device 20 are required during a medical process, e.g. a cataract operation. In order to design an operation efficiently and reduce the changes of the settings to be implemented by a surgeon during the operation, the system 1 described herein is able to allow a surgeon to adapt a medical process, or the associated steps and settings, in advance and able to automatically perform adjustments on the medical device 20 during the operation and able to output instructions for the surgeon, as described below with reference to FIG. 1 and FIG. 2, wherein FIG. 1 depicts the system 1 for configuring at least one medical device 20 and FIG. 2 shows the corresponding method.

The system 1 may be implemented as a single apparatus 2, as depicted in FIG. 1. In an alternative, the various units of the apparatus 2 might also be distributed among various apparatuses and for example be implemented as a cloud service.

The system 1 comprises a transceiver unit 4. This transceiver unit 4 serves firstly for communication with the surgeon, in particular for receiving inputs by the surgeon, and secondly for communication with storage units 8 and the medical devices 20. In this context, the communication, e.g. the data transfer 6 between the transceiver unit 4 and the storage unit 8, may be implemented in wired fashion or wirelessly, e.g. by radio.

Prior to an operation, for example in response to a query by a surgeon, the transceiver unit 4 retrieves predefined steps of a medical process from a storage unit 8. As depicted here in FIG. 1, the storage unit 8 may be implemented as an external database. In an alternative, the storage unit 8 may be integrated in the apparatus 2 and/or be part of the medical device.

As already explained above, a medical process usually consists of multiple steps describing the procedure. Such general predefined steps are stored in the storage unit 8. These steps contain instructions for the surgeon, e.g. acoustic or visual directions or visualizations, and settings of the at least one medical device 20. If a step of the medical process requires multiple medical devices 20, then the corresponding predefined steps contain settings for multiple medical devices 20. These retrieved steps are displayed on a display unit 10 (step S1 in FIG. 2). In particular, the display unit 10 may comprise a graphical user interface for displaying the predefined steps.

In order to avoid time-consuming adjustments during the medical procedure and design the medical procedure or process efficiently, the transceiver unit 4 receives an input 12 by the surgeon prior to the medical process (step S2 in FIG. 2). This input 12 by the surgeon serves to personalize the medical process, and in particular adapt the latter to a patient. For example, the surgeon can alter the sequence of the displayed predefined steps, re-label the displayed predefined steps, adapt settings or parameters of the medical devices 20, select a subgroup of the predefined steps, and the like.

The system 1 also comprises a processing unit 14 designed to adapt the predefined steps on the basis of the input 12 by the surgeon (step S3 in FIG. 2). Adapting the predefined steps implements an adaptation for the specific medical process, in particular a selection of the steps and an adaptation of the parameters for the settings of the at least one medical device 20. The predefined steps contain default values for the at least one medical device 20, and, by way of the input 12, these default values can be adapted on an individual basis for the specific medical process by the surgeon.

Following the adaptation of the predefined steps, the processing unit 14 can work through the adapted steps in sequence during the medical process (step S4 in FIG. 2). Then, instructions for the surgeon based on the adapted steps are output via the display unit 10. For example, the display unit 10 can display the adapted steps and/or the instructions for the surgeon in succession during the medical process. In an alternative, the adapted steps and/or the instructions for the surgeon can be displayed simultaneously, and the respective active step is highlighted.

As already mentioned, the display unit 10 may also be part of the medical device. The instructions, in the form of directions or visualizations, may then be placed over a microscope view, for example as an overlay.

In addition to the instructions for the surgeon, the settings 16 of each adapted step are transferred to the medical device 20 or, in the case of multiple medical devices 20, to each medical device 20. Then, the corresponding parameters for the respective active adapted step are set in the medical device 20.

The transceiver unit 4 is also able to receive an input 12 by the surgeon during the operation. In this case, the input 12 can initiate a changeover between the adapted steps in particular. For example, should it transpire during the operation that a step should be shortened or skipped, then the surgeon can also carry out this adaptation during the operation.

Thus, the system described here renders it possible to already adapt predefined steps for a medical process, in particular adapt these patient specifically, in advance, i.e. prior to the operation itself. This can improve the efficiency during the operation since the surgeon need implement fewer or no adjustments on the medical devices during the operation. To obtain additional flexibility, it is also possible to implement further adaptations still during the operation.

### REFERENCE SIGNS

1 System
2 Apparatus
4 Transceiver unit
6 Data transfer
8 Storage unit
10 Display unit
12 Input
14 Processing unit
16 Settings
20 Medical instrument
S1-S4 Method steps

1-11. (canceled)

12. A system for configuring at least one medical device, the system comprising:
a display unit designed to display predefined steps for at least one medical process, the predefined steps including instructions for a surgeon and settings of the at least one medical device, and the predefined steps comprising default values for the at least one medical device and being based on a default sequence for the at least one medical device,
a transceiver unit designed to receive an input by the surgeon prior to the medical process, and
a processing unit designed to adapt the predefined steps on the basis of the input by the surgeon, the adaptation including a selection of the predefined steps and/or an adaptation of the sequence of the predefined steps, the processing unit being designed to adapt the default values and/or the default sequence on an individual basis for the specific medical process, the processing unit being designed to transfer the adapted steps to the medical device prior to the start of the medical process.

13. The system as claimed in claim 12, wherein the processing unit is designed to work through the adapted steps in sequence during the medical process and, on the basis of the adapted steps, output instructions for the surgeon and configure the settings of the at least one medical device for each adapted step.

14. The system as claimed in claim 12, wherein the transceiver unit is designed to receive an input by the surgeon during the medical process, and wherein the processing unit is designed to further adapt, on the basis of the input by the surgeon, the steps adapted prior to the medical process, the adaptation including a selection of the adapted steps, a sequence of the adapted steps and/or parameters for the settings of the at least one medical device, and/or wherein the input specifies a changeover between the adapted steps.

15. The system as claimed in claim 13, wherein the input by the surgeon specifies a termination and/or a shortening of one of the adapted steps.

16. The system as claimed in claim 12, wherein the adaptation of the predefined steps is patient specific.

17. The system as claimed in claim 12, wherein the display unit comprises a graphical user interface designed to display the predefined steps, the adapted steps, the settings of the at least one medical device and/or the instructions for the surgeon.

18. The system as claimed in claim 12, wherein the display unit is designed to output the instructions for the surgeon visually and/or acoustically.

19. The system as claimed in claim 12, wherein the transceiver unit comprises one or more input devices, in particular a microphone, a mouse, a keyboard, a touchpad and/or a foot switch, and/or is connected to one or more input devices.

20. The system as claimed in claim 19, wherein at least one of the input devices is arranged on the at least one medical device.

21. The system as claimed in claim 12, wherein the system comprises at least one storage unit designed to store the predefined steps and/or the adapted steps with the associated instructions and settings.

22. The system as claimed in claim 21, wherein the at least one storage unit is arranged on the at least one medical device and/or on a server and/or in a cloud.

23. A method for configuring at least one medical device, including the steps of:
displaying predefined steps for at least one medical process, the predefined steps including instructions for a surgeon and settings of the at least one medical device, and the predefined steps comprising default values for the at least one medical device and being based on a default sequence for the at least one medical device,
receiving an input by the surgeon prior to the medical process, and
adapting the predefined steps on the basis of the input by the surgeon, the adaptation including a selection of the predefined steps and/or an adaptation of the sequence of the predefined steps,
with the default values and/or the default sequence being adapted on an individual basis for the specific medical process, the adapted steps being transferred to the medical device prior to the start of the medical process.
